(19) United States
(12) Patent Application Publication
(10) Pub. No.: US 2011/0245641 A1
(43) Pub. Date: Oct. 6, 2011

(54) Monitor With Multi-Position Base

(75) Inventor: Scott MacLaughlin, Windsor, CO (US)

(73) Assignee: Nellcor Puritan Bennett LLC, Boulder, CO (US)

(21) Appl. No.: 13/076,323

(22) Filed: Mar. 30, 2011

Related U.S. Application Data
(60) Provisional application No. 61/319,512, filed on Mar. 31, 2010.

(51) Int. Cl. A61B 5/145; H05K 13/04; H05K 5/00

(52) U.S. Cl. 600/324; 29/592.1; 361/679.01

(57) ABSTRACT

According to various embodiments, a medical monitoring device includes a monitor component and a base component. The base component has one or more connectors on a facing of the base component. The monitor component is capable of rotating with respect to the base component. In various embodiments, the monitor component may be above the base component.

### BACKGROUND

The present disclosure relates generally to medical devices and, more particularly, to medical monitoring devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a medical monitoring device may be used, by a clinician. The device may be connected to a sensor disposed on or in the patient. The front side of the device may have a display, which may show readings obtained by the sensor, and controls, which may enable the clinician to change or adjust measurement settings of the sensor. Thus, it may be important for the clinician to be able to view the front side of the device. Cables connecting the device with the sensor may be coupled to connectors located in a fixed position on the front or side of the device. However, the patient may not be positioned near the front or side of the device. Thus, the cable may be routed a distance from the device to the patient. Because of the bending radius of the cable, the connectors may restrict placement of the device. In addition, in a particular medical setting, the device and/or patient may be moved, which may require that the cables be rerouted or disconnected and reconnected. Providing additional connectors on other sides of the device may be costly and introduce the possibility of confusion as several cables serving different purposes may be connected to the device at the same time.

### BRIEF DESCRIPTION OF THE DRAWINGS

Advantages of the disclosed techniques may become apparent upon reading the following detailed description and upon reference to the drawings in which:

FIG. 1 illustrates a pulse oximetry system coupled to a multi-parameter patient monitor and a sensor according to various embodiments;

FIG. 2 illustrates a simplified block diagram of a pulse oximeter in FIG. 1, according to an embodiment;

FIG. 3 is a perspective view of an exemplary medical monitoring device with a base component and a monitor component that rotate with respect to each other, according to an embodiment;

FIG. 4 is a perspective view of an exemplary medical monitoring device with a bearing assembly, according to an embodiment;

FIG. 5 is a perspective view of an exemplary medical monitoring device with a slip ring assembly, according to an embodiment;

FIG. 6 is a perspective view of an exemplary medical monitoring device showing the base component rotated 90 degrees, according to an embodiment;

FIG. 7 is a perspective view of an exemplary medical monitoring device showing the base component rotated 45 degrees, according to an embodiment; and

FIG. 8 is a perspective view of an exemplary medical monitoring device located next to a patient with the base component rotated 90 degrees, according to an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A clinician may use a medical monitoring device, such as a pulse oximeter, to monitor certain aspects of the condition of a patient. The aspects may be determined using a sensor coupled either externally or internally to the patient. Examples of monitored parameters may include body temperature, pulse rate, respiration rate, blood pressure, blood oxygenation, or electrical activity. Other parameters may be monitored depending on the condition of the patient. Signals from the sensor may be sent to the monitoring device via an electrical or optical conductor, such as a cable, connecting the sensor and the device. In addition, signals from the monitoring device may pass through the electrical or optical conductor to the sensor. Some sensors may require power that is provided via the monitoring device. In one embodiment, the cable housing the conductor may be composed of several layers, which may include shielding to prevent electrical or optical interference and armor or braiding to protect the conductor from physical damage and/or abrasion. The stiffness of the conductive elements themselves, as well as such additional layers may make it difficult to bend the cable, resulting in a large bending radius. In addition, the connectors on the end of the cable interfacing with the device may be different depending on the type of sensor. The monitoring device may be configured to monitor more than one aspect of the patient, thus it may have a variety of different connectors to enable it to couple with different sensors. Finally, the monitoring device may connect to a power source via a connected power cable.

A medical monitoring device may be used in a variety of settings, which may include operating rooms, intensive care units, recovery rooms, general care floors, and examination rooms. Depending on the particular circumstances, the device, the patient, or both may be moved. For example, a single device may be moved from room to room to take periodic measurements of multiple patients. Moreover, a device may be moved from the side of a patient onto a gurney used to move the patient to another room. Thus, the optimal routing of cables between the device and patient may change often or rapidly as the patient and/or device are moved about. Many considerations affect the optimal routing of cables and may include the specific type of medical environment, space constraints, clinician or patient preference, ease of access, patient comfort, visibility of the device by the clinician, reducing interference with high-traffic or high-access areas, whether the patient or clinician is right or left handed, reducing interference with clinician tasks, or reducing interference with other objects, such as intravenous lines. Fixed positions of connectors on the device may not provide the flexibility to accommodate the varied and frequently changing situations and needs in different medical settings. For example, connectors attached to the front of the device may obscure the device display, inhibit access to buttons or knobs, and may detract from the aesthetics of the device. Moreover, connecting to the side of the device may pose other disadvantages, such as not being able to place other equipment (e.g., monitors, pumps, treatment devices, etc.) beside the device because of a large bend radius associated with a connected cable.

In certain embodiments described below, the medical monitoring device consists of a monitor component and a base component, which together enable connectors on the base component to be rotated into convenient positions for cable routing. As situations change or as the device or patient moves, the base component may be rotated to maintain or adjust the desired routing of the cables. The device may be located anywhere it is needed, which may include on a table top, mounted on a pole or wall, or placed on the gurney of the patient. For example, the device may be located on top of or in between other devices located on a cart. The base component may be coupled to the bottom, top, or any other side of the monitor component. Thus, the base and monitor components may enable movement of the device, patient, or both without inconvenient routing of cables or having to disconnect and reconnect cables.

In certain embodiments, the disclosed medical monitoring devices, systems, and methods may be used in conjunction with monitoring of any appropriate medical aspect, such as, but not limited to temperature, pulse rate, respiration rate, blood pressure, blood oxygenation (pulse oximetry), or electrical activity. The present techniques may also be used on devices used to treat any patient connected to any medical device. Further, the devices and techniques provided herein may be used to treat human patients, such as trauma victims, anesthetized patients, cardiac arrest victims, patients suffering from airway obstructions, and patients suffering from respiratory failure.

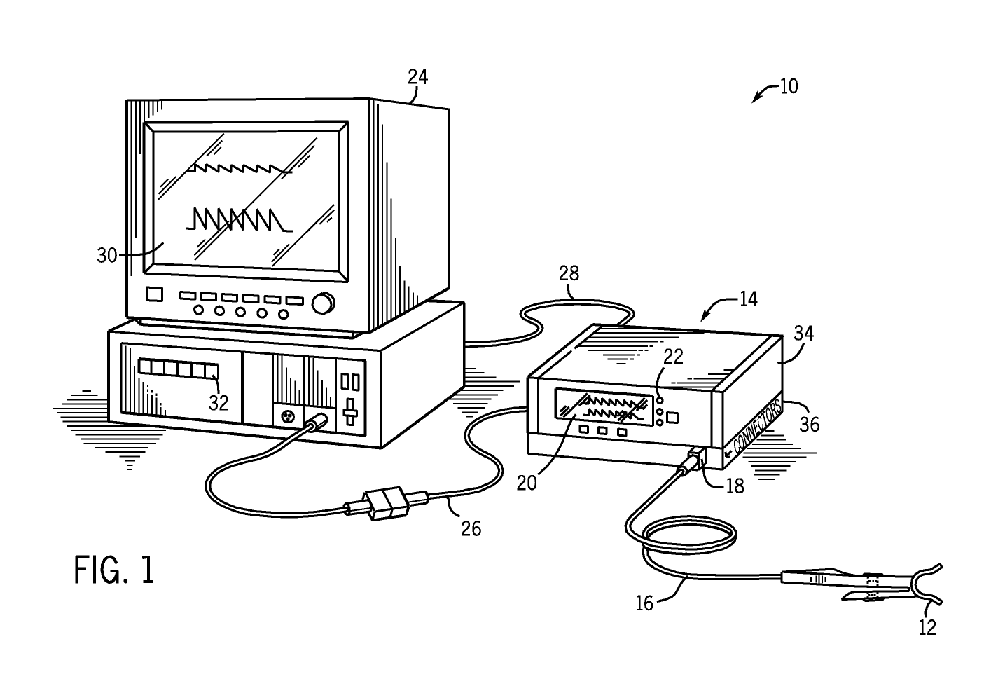

One embodiment of a monitor including a base component is depicted in FIG. 1. In particular, FIG. 1 depicts a medical monitoring system 10 having a sensor 12 coupled to a monitor 14 in accordance with an embodiment of the present disclosure. The sensor 12 may be coupled to the monitor 14 via sensor cable 16 and sensor connector 18. The monitor 14 may be any suitable monitor, such as those available from Nellcor Puritan Bennett, LLC. The monitor 14 may be configured to calculate physiological parameters from signals received from the sensor 12 when the sensor 12 is placed on a patient. In some embodiments, the monitor 14 may be primarily configured to determine, for example blood and/or tissue oxygenation and perfusion, respiratory rate, respiratory effort, continuous non-invasive blood pressure, cardiovascular effort, glucose levels, level of consciousness, total hematocrit, hydration, electrocardiography, temperature, or any other suitable physiological parameter. Additionally, the monitor 14 may include a display 20 configured to display information regarding the physiological parameters, information about the system, and/or alarm indications. The monitor 14 may include various input components 22, such as knobs, switches, keys and keypads, buttons, etc., to provide for operation and configuration of the monitor.

Furthermore, to upgrade conventional operation provided by the monitor 14 to provide additional functions, the monitor 14 may be coupled to a multi-parameter patient monitor 24 via a cable 26 connected to a sensor input port or via a cable 28 connected to a digital communication port. In addition to the monitor 14, or alternatively, the multi-parameter patient monitor 24 may be configured to calculate physiological parameters and to provide a central display 30 for information from the monitor 14 and from other medical monitoring devices or systems. In some embodiments, the monitor 24 may be primarily configured to display and/or determine some or all of the same physiological parameters as monitor 14. The monitor 24 may include various input components 32, such as knobs, switches, keys and keypads, buttons, etc., to provide for operation and configuration of the monitor 24. In addition, the monitor 14 and/or the multi-parameter patient monitor 24 may be connected to a network to enable the sharing of information with servers or other workstations.

The sensor 12 may be any sensor suitable for detection of any physiological parameter. The sensor 12 may include optical components (e.g., one or more emitters and detectors), acoustic transducers or microphones, electrodes for measuring electrical activity or potentials (such as for electrocardiography), pressure sensors, motion sensors, temperature sensors, etc. In one embodiment, the sensor 12 may be configured for photo-electric detection of blood and tissue constituents. For example, the sensor 12 may be a pulse oximetry sensor, such as those available from Nellcor-Puritan Bennett. As shown in FIG. 1, the sensor 12 may be a clip-type sensor suitable for placement on an appendage of a patient, e.g., a digit, an ear, etc. In other embodiments, the sensor 12 may be a bandage-type sensor having a generally flexible sensor body to enable conformable application of the sensor to a sensor site on a patient. In yet other embodiments, the sensor 12 may be secured to a patient via adhesive (e.g., in an embodiment having an electrode sensor) on the underside of the sensor body or by an external device, such as headband or other elastic tension device. In yet other embodiments, the sensor 12 may be configurable sensors capable of being configured or modified for placement at different sites (e.g., multiple tissue sites, such as a digit, a forehead of a patient, etc.).

Returning to the monitor 14 shown in FIG. 1, the monitor 14 may be divided into two connected sections: a monitor component 34 and a base component 36. In one embodiment, the monitor component 34 may include the display 20 and input components 22 and the base component 36 may include the sensor connector 18. The monitor component 34 may be mounted above the base component 36. As will be discussed in more detail below, the monitor component 34 is capable of rotating with respect to base component 36 to enable either the display 20 to face in different directions or to enable the sensor cable 16 to be routed differently.

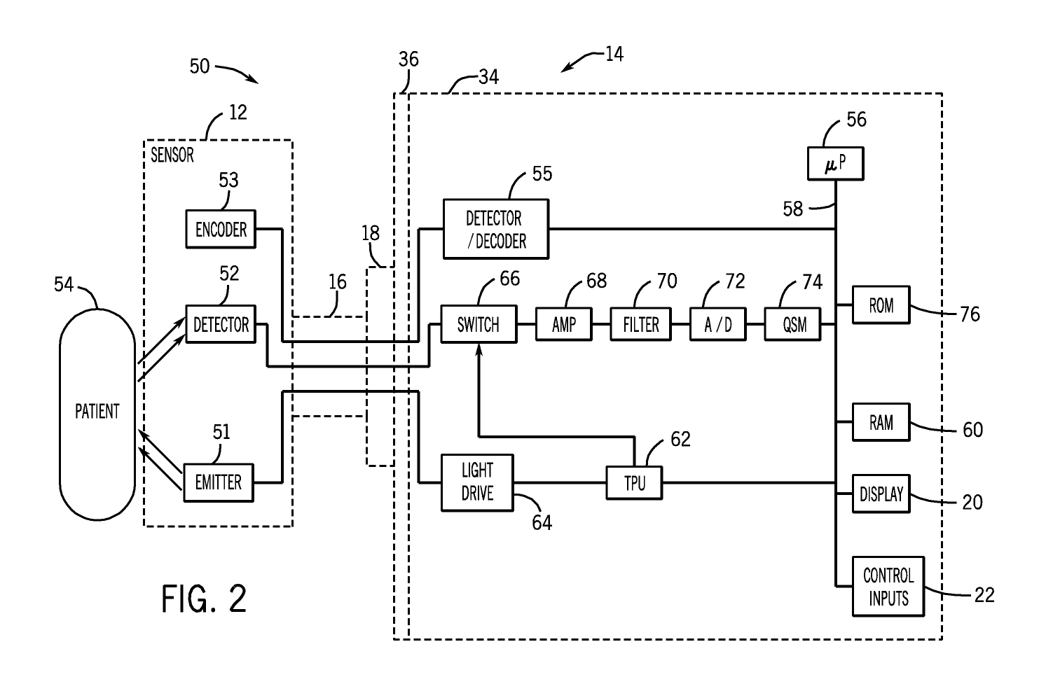

Turning to FIG. 2, a simplified block diagram 50 of a pulse oximeter is illustrated in accordance with an embodiment. Specifically, certain components of the sensor 12 and the monitor 14 are illustrated in FIG. 2. The sensor 12 may include an emitter 51, a detector 52, and an encoder 53. It should be noted that the emitter 51 may be capable of emitting at least two wavelengths of light, e.g., RED and infrared (IR) light, into the tissue of a patient 54, where the RED wavelength may be between about 600 nanometers (nm) and about 700 nm, and the IR wavelength may be between about 800 nm and about 1000 nm. The emitter 51 may include a single emitting device, for example, with two LEDs or the emitter 51 may include more than one emitting device with, for example, multiple LEDs at various locations. Regardless of the number of emitting devices, the emitter 51 may be used to measure, for example, water fractions, hematocrit, or other physiologic parameters of the patient 54. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 52 may be an array of detector elements that may be capable of detecting light at various intensities and wavelengths. In operation, light enters the detector 52 after passing through the tissue of the patient 54. The detector 52 may convert the light at a given intensity, which may be directly related to the absorbance and/or reflectance of light in the tissue of the patient 54, into an electrical signal. That is, when more light at a certain wavelength is absorbed or reflected, less light of that wavelength is typically received from the tissue by the detector 52. For example, the detector 52 may include one or more photodiodes, or any other element capable of converting light into either a current or voltage. After converting the received light to an electrical signal, the detector 52 may send the signal to the monitor 14, where physiological characteristics may be calculated based at least in part on the absorption of light in the tissue of the patient 54.

Additionally the sensor 12 and/or sensor cable 16 may include an encoder 53, which may contain information about the sensor 12, such as what type of sensor it is (e.g., whether the sensor is intended for placement on a forehead or digit) and the wavelengths of light emitted by the emitter 51. This information may allow the monitor 14 to select appropriate algorithms and/or calibration coefficients for calculating the physiological characteristics of the patient 54. The encoder 53 may, for instance, be a memory on which one or more of the following information may be stored for communication to the monitor 14: the type of the sensor 12; the wavelengths of light emitted by the emitter 51 and the proper calibration coefficients and/or algorithms to be used for calculating the physiological characteristics of the patient 54. In one embodiment, the data or signal from the encoder 53 may be decoded by a detector/decoder 55 in the monitor 14.

Signals from the detector 52 and the encoder 53 may be transmitted to the monitor 14. In one embodiment, the signals pass through electrical and/or optical conductors that pass through the sensor cable 16, sensor connector 18, and base component 36, before terminating in the monitor component 34. In one embodiment, the electrical and/or optical connections remain unchanged by rotation of the monitor component 34 with respect to the base component 36. In other words, the clinician may rotate the monitor component 34 without affecting the internal connections of the electrical and/or optical conductors.

The monitor 14 may include one or more processors 56 coupled to an internal bus 58. Also connected to the bus may be a RAM memory 60 and a display 20. A time processing unit (TPU) 62 may provide timing control signals to light drive circuitry 64, which controls when the emitter 51 is activated, and if multiple light sources are used, the multiplexed timing for the different light sources. TPU 62 may also control the gating-in of signals from detector 52 through a switching circuit 66. These signals are sampled at the proper time, depending at least in part upon which of multiple light sources is activated, if multiple light sources are used. The received signal from the detector 52 may be passed through an amplifier 68, a low pass filter 70, and an analog-to-digital (A/D) converter 72 for amplifying, filtering, and digitizing the electrical signals the from the sensor 12. The digital data may then be stored in a queued serial module (QSM) 74, for later downloading to RAM 60 as QSM 74 fills up. In an embodiment, there may be multiple parallel paths for separate amplifiers, filters, and AID converters for multiple light wavelengths or spectra received.

In an embodiment, based at least in part upon the received signals corresponding to the light received by detector 52, the processor 56 may calculate the oxygen saturation using various algorithms. These algorithms may require coefficients, which may be empirically determined. For example, algorithms relating to the distance between the emitter 51 and various detector elements in the detector 52 may be stored in a ROM 76 and accessed and operated according to processor 56 instructions.

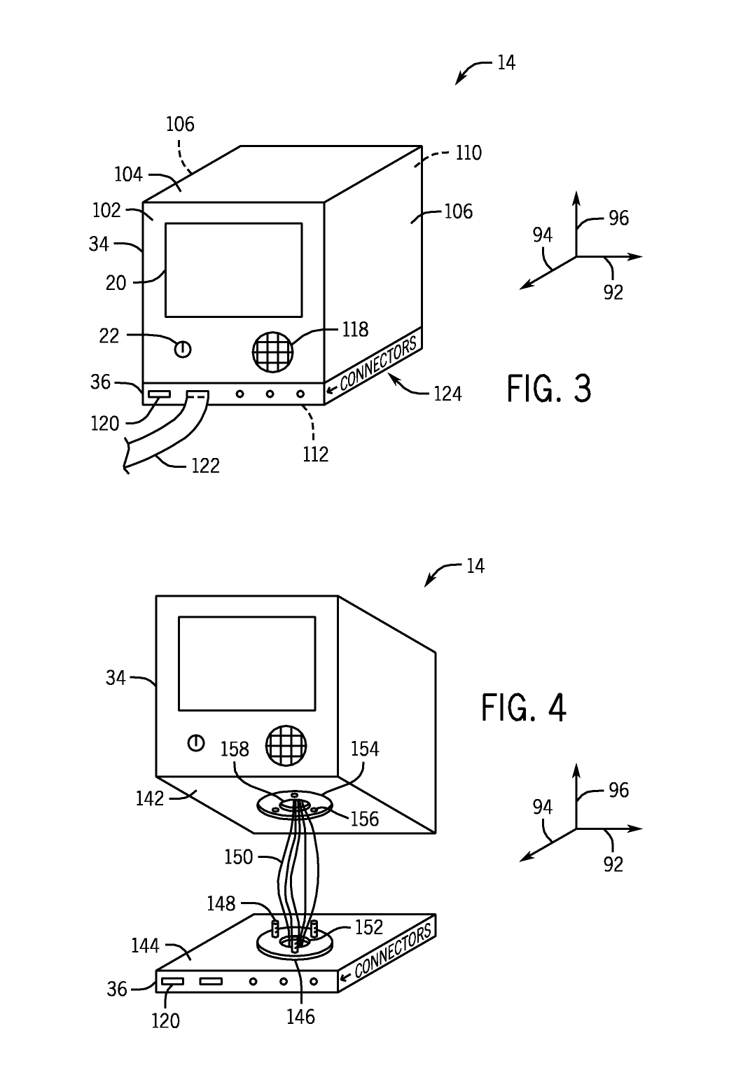

With the preceding in mind, FIG. 3 is a perspective view of the monitor 14 in accordance with an embodiment of the present disclosure. A coordinate system with an x-axis 92, a y-axis 94, and a z-axis 96 is shown. In the illustrated embodiment, the monitor component 34 is mounted above the base component 36 and the monitor component is capable of rotating about the z-axis 96. The system has a front 102, a top 104, sides 106, a back 110, and a bottom 112. The monitor component 34 may include a touchscreen or display 20 to provide information to a user and/or allow for input. In addition, the monitor component 34 may have one or more input components 22 for user input or selection. The monitor component 34 may also have a speaker 118 to provide audio feedback. The display 20, input components 22, and speaker 118 may be located on the front 102 of the monitor component 34. The front 102 of the base component 36 may include one or more input, output, or power connectors 120 for the monitor 14. Attached to the connectors 120 may be one or more cables 122 connected to medical devices or sensors or to power sources. Finally, a legend 124, such as the text “Connectors” along with an arrow pointing to the front 102 or the text “Connectors located to the left,” and/or a symbol representative of the connector along with an arrow pointing to the front, may be disposed on the appropriate sides of the base component 36. If the front 102 of the base component 36 has been rotated, the legend 124 enables a user looking at the front of the monitor 14 to quickly locate the connectors 120.

FIG. 4 is a perspective view of one embodiment of the monitor 14 showing a monitor component 34 that may be coupled to the base component 36 via a bearing assembly 146. The monitor component 34 has an internal bottom 142 and the base component 36 has an internal top 144. The bearing assembly 146 enables the base component 36 to rotate about the z-axis 96. The bearing assembly 146 may consist of a lower plate and an upper plate separated by ball bearings. In other embodiments, other configurations common to bearing assemblies 146 may also be used. The bearing assembly 146 is coupled to the base component 36 using any common method, such as screwed fasteners, welding, or other suitable techniques for mechanically affixing two structures. Studs 148 may be attached to the upper plate of the bearing component 146 to enable coupling with the monitor component 34. Internal conductors 150, e.g. wires, pass between the monitor component 34 and the base component 36 and may be routed through a hole 152 in the bearing assembly 146. For each internal conductor 150, one end is coupled internally to the monitor component 34 and another end is coupled internally to the connector 120. Enough slack may be provided in the conductors 150 to enable rotation of the base component 36 clockwise or counterclockwise from a starting point with connectors 120 on the front 102. In one embodiment, the base component 36 may be rotated 90 degrees, 180 degrees, or more. Alternatively, the conductors 150 may be flexible enough to accommodate rotation. A recessed area 154 may be provided in the bottom 142 of the monitor component 34 to fit the bearing component 146. Thus, only a small gap may exist between the monitor component 34 and the base component 36 when coupled. Holes 156 may be disposed in the recessed area 154 to mate with the studs 148 to enable the monitor component 34 to be coupled to the bearing assembly 146. A hole 158 may be provided in the recessed area 154 to enable the conductors 150 to pass into the monitor component 34, Alternatively, the bearing assembly 146 may be coupled to the monitor component 34 and the recessed area 154 provided in the base component 36. Besides bearing and slip ring assemblies, other methods of coupling the monitor component 34 and base component 36 such that rotation is enabled, such as pivots, swivels, or ball joints, may also be used.

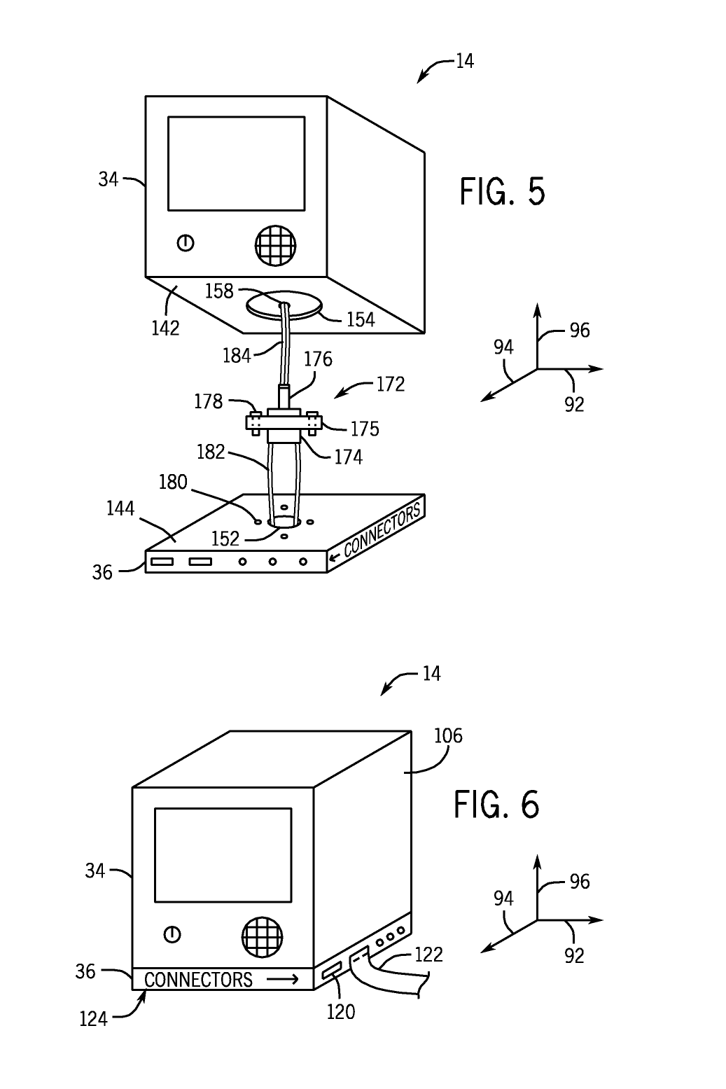

FIG. 5 is a perspective view of one embodiment of a monitor 14 showing a monitor component 34 that may be coupled to the base component 36 via a slip ring assembly 172. The slip ring 172 enables the base component 36 to rotate about the z-axis 96. The slip ring 172 may consist of a lower component 174 and an upper component 176. Other configurations common to slip rings 172 may also be used. The lower component 174 may be coupled to the base component 36 via bolts 178 passing through holes of a mounting flange 175 and screwed into holes 180 in the base component. Other common methods of fastening, such as welding, may also be used. Similarly, the upper component 176 is coupled to the monitor component 34. The mounting flange 175 of the slip ring 172 may rest on the top 144 of the base component 36 and fit into a recessed area 154 in the bottom 142 of the monitor component 34, such that only a small gap may exist between the monitor component and the base component when coupled. Alternatively, the recessed area 154 may be provided in the top 144 of the base component 36. A lower set of conductors 182 connects the lower component 174 to the base component 36 and an upper set of conductors 184 connects the upper component 176 to the monitor component 34. In one embodiment, the slip ring 172 is configured such that electrical continuity exists between corresponding pairs of upper and lower conductors during rotation. In other embodiments, a fiber optic rotary joint (similar to a slip ring) may be used to maintain continuity of optical signals. Thus, in this embodiment, no additional length or flexibility of the conductors is required.

FIG. 6 is a perspective view of the monitor 14 with the base component 36 rotated 90 degrees counterclockwise when viewed looking down along the z-axis 96. In other words, the connectors 120 and cables 122 are located on a side 106 of the monitor 14. Such an orientation of the cables 122 may be advantageous if the patient is located to the side of the monitor 14. In addition, the legend 124 is located on the front 102 where a clinician may be looking when using the monitor 14. Thus, if the clinician could not easily see where the connectors 120 were located, the legend 124 would indicate where the clinician should look to find them. In other embodiments, the base component 36 may be rotated 90 degrees clockwise or rotated 180 degrees.

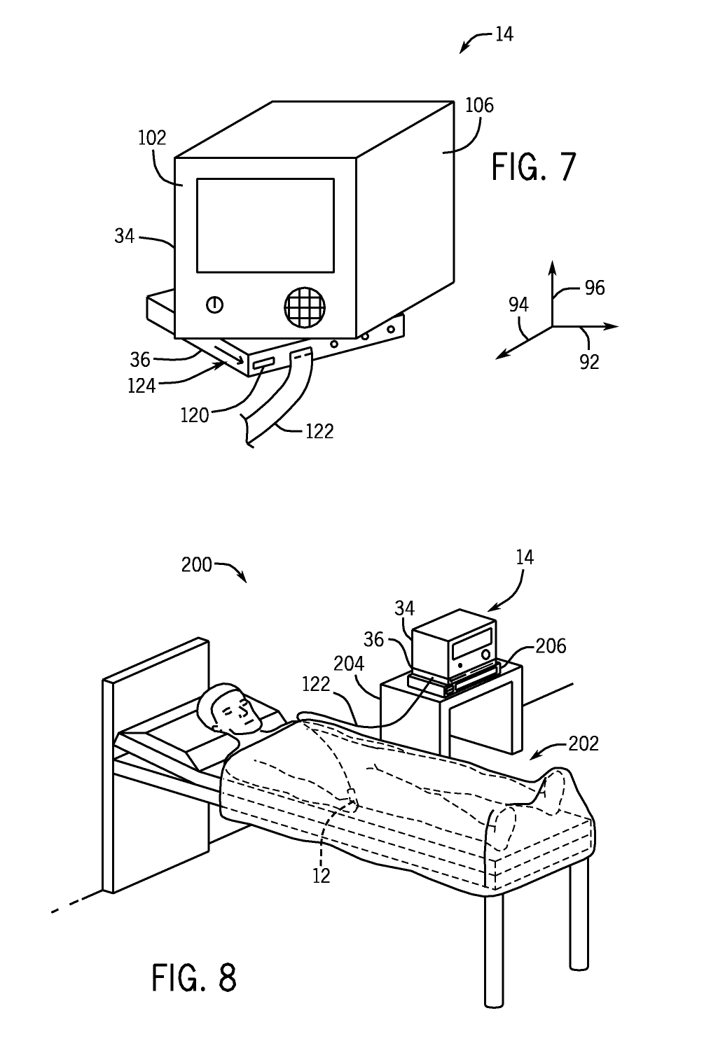

FIG. 7 is a perspective view of the monitor 14 with the base component 36 rotated to 45 degrees counterclockwise when viewed looking down along the z-axis 96. Such an orientation of the connectors 120 may be advantageous if the patient is not located directly to the sides 106 or the front 102 of the monitor 14. In the embodiment shown, the legend 124 is still visible to a user standing in front of the monitor 14 to indicate where the connectors 120 are located. A detent mechanism or a similar mechanism, such as a catch or spring-operated mechanism, may be incorporated into either the bearing component 146 or the slip ring component 172, such that the base component 36 snaps into positions located at less than 90-degree increments. For example, the increments may be 30 degrees, 45 degrees, or any other convenient interval. The detents may enable the base component 36 to hold itself in position until physically rotated by the clinician. Thus, unintentional events, such as tugs on the cables 122 or bumps into the base component 36 or monitor component 34, may not easily move the base or monitor components out of position.

FIG. 8 is a perspective view of a pulse oximetry system 200 used to monitor a patient 202 lying on a bed. The monitor component 34 and base component 36 of the pulse oximeter monitor 14 may rest on top of one or more other medical devices 206, which in turn may rest on a table, cart, stand, or shelf 204. Connected to the base component 36 is a pulse oximetry sensor 12, which is disposed on the finger of the patient 202. In the particular embodiment shown, the front 102 of the base component 36 is rotated 90 degrees counterclockwise relative to the front of the monitor component 34. Such an orientation enables the cable 122 to be routed from the base component 36 to the patient 202 without getting in the way of the clinician or the patient. If the monitor 14 was on the other side of the patient 202, the front 102 of the base component 36 may be rotated 90 degrees clockwise relative to the front of the monitor component 34. Similarly, if the monitor 14 was at the foot of the bed, the front 102 of the base component 36 may be rotated 180 degrees relative to the front of the monitor component 34. In addition, the monitor 14 may be placed on the bed alongside or on top of the patient 202 if the bed or gurney were to be moved. Then the base component 36 may be rotated in any direction that is convenient for the clinician and/or patient 202. Other configurations and degrees of rotation may be possible depending on the requirements of a particular patient 202 or clinician.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

What is claimed is:

1. A medical monitoring device comprising:
a monitor component; and
a base component, comprising one or more connectors on a facing of the base component;
wherein the monitor component is capable of rotating with respect to the base component.

2. The medical monitoring device of claim 1, wherein a bearing assembly couples the monitor component and base component together and enables rotation.

3. The medical monitoring device of claim 2, further comprising:
at least one internal conductor, wherein a first end is coupled internally to the monitor component and a second end is coupled internally to at least one connector disposed on the base component.

4. The medical monitoring device of claim 2, wherein the monitor component is capable of rotating up to 180 degrees with respect to the base component.

5. The medical monitoring device of claim 1, wherein a slip ring assembly couples the monitor component and base component together, enables the monitor component to rotate with respect to the base component, and maintains electrical continuity between the monitor component and base component.

6. The medical monitoring device of claim 1, wherein a fiber optic rotary joint couples the monitor component and base component together, enables the monitor component to rotate with respect to the base component, and maintains continuity of optical signals between the monitor component and base component.

7. The medical monitoring device of claim 1, wherein a detent mechanism enables the monitor component to be held in place at increments of 30 degrees or 45 degrees.

8. The medical monitoring device of claim 1, wherein an external device coupled to a respective connector on a facing of the base component via a cable receives input from the monitor component, transmits output to the monitor component, receives power from the monitor component, or any combination thereof.

9. The medical monitoring device of claim 1, wherein the base component further comprises a legend disposed on at least one facing of the base component indicating where the one or more connectors are located.

10. A pulse oximetry system comprising:
a pulse oximetry monitor capable of receiving signals generated by a pulse oximetry sensor, when present, the pulse oximetry monitor comprising:
a monitor component; and
a base component that rotates with respect to the monitor component, the base component comprising one or more connectors on a facing of the base component.

11. The pulse oximetry system of claim 10, wherein the pulse oximetry sensor, when present, receives signals from the pulse oximetry monitor via a cable connected to a respective connector of the one or more connectors.

12. The pulse oximetry system of claim 10, wherein text and/or symbols disposed on at least one facing of the base component indicate where the one or more connectors are located.

13. The pulse oximetry system of claim 10, wherein the monitor component is capable of rotating up to 180 degrees with respect to the base component.

14. The pulse oximetry system of claim 10, wherein detents or a catch and/or spring-operated mechanism holds the base component with respect to the monitor component in positions at increments of 30 degrees or 45 degrees.

15. The pulse oximetry system of claim 10, wherein a bearing assembly, slip ring assembly, fiber optic rotary joint, pivot, swivel, ball joint, or a combination thereof couples the monitor component and base component together and enables rotation.

16. A method of manufacturing a medical monitor, the method comprising:
coupling or attaching a monitor component and base component together, such that the monitor component and base component rotate with respect to one another;
providing one or more connectors on a facing of the base component; and
providing an electrical and/or optical connection between the one or more connectors on the facing of the base component and one or more internal components of the monitor component.

17. The method of claim 16, wherein coupling or attaching the monitor component and base component together comprises using bearings, a slip ring, a fiber optic rotary joint, a pivot, a swivel, a ball joint, or a combination thereof.

18. The method of claim 16, further comprising selecting at least one of the length or flexibility of the electrical and/or optical connection such that the monitor component is capable of rotating up to 180 degrees with respect to the base component.

19. The method of claim 16, further comprising providing a legend on at least one facing of the base component indicating where the one or more connectors are located.

20. The method of claim 16, further comprising providing a detent mechanism between the base component and monitor component to enable the monitor component to be held in place at increments of 30 degrees or 45 degrees.
